(19) United States
(12) Patent Application Publication
(10) Pub. No.: US 2023/0405351 A1
(43) Pub. Date: Dec. 21, 2023

(54) STERILIZABLE CATHETER AND LIGHT FIBER FOR USE THEREWITH AND METHODS OF STERILIZATION

(71) Applicant: Alucent Biomedical Inc., Salt Lake City, UT (US)

(72) Inventors: James D. ISAACSON, Salt Lake City, UT (US); RB Eugene HAYES, Salt Lake City, UT (US); Dylan James BLAIR, Salt Lake City, UT (US)

(21) Appl. No.: 18/332,643

(22) Filed: Jun. 9, 2023

Related U.S. Application Data
(60) Provisional application No. 63/351,268, filed on Jun. 10, 2022.

(51) Int. Cl. A61N 5/06; A61L 2/08

(52) U.S. Cl. CPC A61N 5/062; A61N 5/0601; A61L 2202/24; A61N 2005/0602; A61L 2/087

(57) ABSTRACT

An apparatus includes a catheter including light fibers extending from a proximal section to a distal section. The apparatus may include an emission section positioned on the distal section that is coated with an NVS agent. The material of the emission section may be different from the other sections of the catheter. The apparatus can be sterilized using E-beam sterilization.

### CROSS-REFERENCE TO RELATED APPLICATIONS

This application claims the benefit of priority of U.S. Provisional Patent Application No. 63/351,268, filed on Jun. 10, 2022, the entirety of which is incorporated by reference herein.

### BACKGROUND

### Technical Field

Systems and methods consistent with the present disclosure provides novel catheters. More particularly, the catheters including light fibers can be sterilized using E-beam sterilization.

### Background Description

Natural Vascular Scaffolding (NVS) Therapy refers to treatment of a substrate using at least one active agent according to the present disclosure. NVS interlinks collagen and elastin by covalently linking these proteins via photoactivation. Catheters can be employed to deliver NVS compounds to a treatment site within the body. These catheters can also include a light fiber to deliver light to the treatment site for photoactivation of the NVS agent.

Incorporation of light fibers are into catheters used for NVS therapy present a complication to the required sterilization of these catheters. Two common sterilization processes are electron beam (or E-beam) sterilization and ethylene oxide (EtO) sterilization. E-beam sterilization uses an electron beam to ionize the surface of a material to sterilize it. EtO sterilization uses ethylene oxide gas, usually under low pressure or under vacuum. For many medical devices, sterilization with ethylene oxide is chosen because it does not damage the device during the sterilization process. Medical devices made from certain polymers, plastics, resins, metals, or glass benefit from the non-reactive nature of EtO. The permeability of gas into difficult to access features such as the lumens of catheters are also usually sterilized using ethylene oxide.

Alucent's current NVS drug-coated balloon catheter is sterilized using E-Beam sterilization, in part because the NVS drug cannot retain its required specifications after experiencing EtO sterilization. The light fiber used to activate the drug, on the other hand, is currently EtO sterilized, because radiation sterilization degrades the core (plastic) light fiber material and affects its optical properties, to the point where there is not adequate light transmission through the fiber and delivery catheter (which may include a balloon) to the drug for the desired level or rate of photoactivation.

The separate sterilization processes for the catheter and drug on the one hand and the light fiber on the other is time consuming and expensive. Further, it would be more efficient to be able to sterilize the catheter and light fiber together after they have been joined.

### SUMMARY

The current disclosure includes an exemplary concept to aid in the integration of an optical light fiber into a drug-coated (e.g., NVS-coated) balloon catheter such that the entire assembly may be E-beam sterilized (or possibly gamma radiation-based sterilized) while retaining acceptable optical performance.

Through various prototyping and studies, it was found that plastic fibers (material that is typically impacted by E-beam sterilization) are less impacted when the core fiber diameter is decreased. NVS catheters in the art have light-fiber configurations composed of 100% PMMA (plastic) at a relatively large core diameter of 0.5 mm. Decreasing the core diameter by half, to 0.25 mm, increases the transmission by about 30×.

According to the present disclosure, other possible approaches to achieve an acceptable light transmission through a light fiber that has been E-beam sterilized include configurations for splicing or connecting different fiber optic materials (e.g. silica and plastic) in series, with the understanding that silica can withstand E-beam irradiation better than plastic. It should also be noted that it is currently understood that Alucent's application requires the use of plastic for its radial emission zone or diffuser portion of the fiber, where the drug is activated.

In addition to employing different material combinations, further embodiments according to the present disclosure include sterilizing smaller core coated light fibers as supplied by Medlight and Pioneer Optics). Preliminary results indicate a significant improvement in the light transmission through a smaller core plastic fiber versus a larger core plastic fiber post E-beam sterilization, suggesting the smaller core diameter help resist optical degradation experienced by lager core fibers after E-beam sterilization.

The resistance to damage demonstrated by coated fibers can advantageously permit sterilizing the fiber in combination with the catheter after the fiber is placed within the catheter using E-beam sterilization.

### BRIEF DESCRIPTION OF THE DRAWINGS

The accompanying drawings, which are incorporated in and constitute a part of this disclosure, illustrate various embodiments and aspects of the present disclosure. In the drawings:

FIG. 1 is a side elevational view of an exemplary apparatus including a catheter, according to embodiments of the present disclosure.

FIG. 2 is a side elevational view of a light fiber of the exemplary catheter of FIG. 1.

FIG. 3 is a side elevational view of a distal portion of the light fiber of FIG. 2.

### DETAILED DESCRIPTION

The following detailed description refers to the accompanying drawings. Wherever possible, the same reference numbers are used in the drawings and in the following description to refer to the same or similar parts. While several exemplary embodiments and features of the invention are described herein, modifications, adaptations, and other implementations are possible without departing from the spirit and scope of the disclosure. For example, substitutions, additions, or modifications may be made to the components illustrated in the drawings, and the exemplary methods described herein may be modified by substituting, reordering, or adding steps to the disclosed methods. Accordingly, the following detailed description does not limit the disclosure. Instead, the proper scope of the disclosure is defined by the appended claims.

As used herein, the following definitions shall apply unless otherwise indicated.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B”, when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein “Natural Vascular Scaffolding (NVS) Therapy” refers to treatment of a substrate using at least one active agent according to the present disclosure. NVS interlinks collagen and elastin by covalently linking these proteins via photoactivation.

The “at least one active agent” is chosen from dimeric naphthalimide compounds. Certain dimeric naphthalimide compounds have been previously disclosed. See, e.g., U.S. Pat. No. 6,410,505 B2. For example, a dimeric naphthalimide compound, 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione), also known as 10-8-10 dimer, 6-[2-[2-(2-aminoethoxy)ethoxy]ethylamino]-2-[2-[2-[2-[6-[2-[2-(2-aminoethoxy)ethoxy]ethylamino]-1,3-dioxobenzo[de]isoquinolin-2-yl]ethoxy]ethoxy]ethyl]benzo[de]isoquinoline-1,3-dione; 2,2′-[1,2-ethanediylbis(oxy-2,1-ethanediyl)]bis[6-({2-[2-(2-aminoethoxy)ethoxy]ethyl}amino)-1H-benzo[de]isoquinoline-1,3(2H)-dione]; and 1H-benz[de]isoquinoline-1,3(2H)-dione, 2,2′-[1,2-ethanediylbis(oxy-2,1-ethanediyl)]bis[6-[[2-[2-(2-aminoethoxy)ethoxy]ethyl]amino]-(9C1), and herein referred to as Compound of Formula (I), has been disclosed. Id.

In some embodiments, the at least one active agent is chosen from the Compound for Formula (I) and pharmaceutically acceptable salts thereof. The “Compound of Formula (I)” as used herein may be described by the structure:

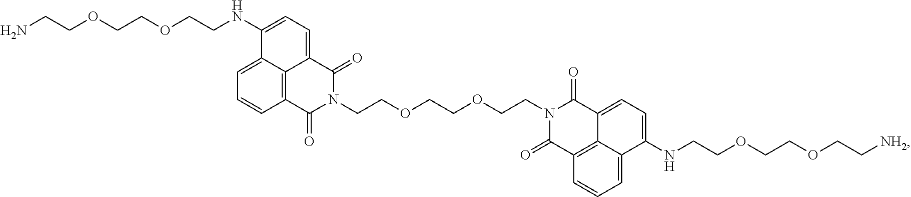 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

by the chemical names 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione); 6-[2-[2-(2-aminoethoxy)ethoxy]ethylamino]-2-[2-[2-[2-[6-[2-[2-(2-aminoethoxy)ethoxy]ethylamino]-1,3-dioxobenzo[de]isoquinolin-2-yl]ethoxy]ethoxy]ethyl]benzo[de]isoquinoline-1,3-dione; 2,2′-[1,2-ethanediylbis(oxy-2,1-ethanediyl)]bis[6-({2-[2-(2-aminoethoxy)ethoxy]-ethyl}amino)-1H-benzo[de]isoquinoline-1,3(2H)-dione]; or 1H-benz[de]isoquinoline-1,3(2H)-dione, 2,2′-[1,2-ethanediylbis(oxy-2,1-ethanediyl)]bis[6-[[2-[2-(2-aminoethoxy)ethoxy]ethyl]amino]-(9C1), or by the Chemical Abstract Services (CAS) Registry No. 438200-66-9.

As used herein, “Compound of Formula (I)” includes one or more of the conformational forms of the compound. Unless stated otherwise, compounds depicted herein coexisting with tautomeric forms are within the scope of the disclosure. Additionally, unless stated otherwise, structures depicted herein are also meant to include compounds that differ only in the presence of one or more isotopically enriched atoms. For example, compounds having the depicted structures except for the replacement of hydrogen by deuterium or tritium, or the replacement of a carbon atom by ¹³C- or ¹⁴C-enriched carbon atom are within the scope of this disclosure.

The at Least One Active Agent

In some embodiments, the at least one active agent is chosen from dimeric naphthalimides and pharmaceutically acceptable salts thereof, e.g., the dimeric naphthalimides disclosed in U.S. Pat. No. 6,410,505 B2. In some embodiments, the at least one active agent is a Compound of Formula (I). In some embodiments, the at least one active agent is chosen from a Compound of Formula (I) and pharmaceutically acceptable salts thereof.

In some embodiments, the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate. In some embodiments, the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium).

Compositions

In some embodiments, the methods provided herein comprise administration of a composition comprising at least one active agent to a natural vessel or AVF. In some embodiments, the at least one active agent is present in an amount ranging from 0.01% to 5% by weight of the composition. In some embodiments, the at least one active agent is present in an amount ranging from 0.01% to 4% by weight of the composition. In some embodiments, the at least one active agent is present in an amount ranging from 0.01% to 2.5% by weight of the composition. In some embodiments, the at least one active agent is present in an amount of 0.01%, 0.03%, 0.05%, 0.07%, 0.09%, 0.11%, 0.13%, 0.15%, 0.17%, 0.19%, 0.21%, 0.23%, 0.25%, 0.27%, 0.29%, 0.31%, 0.33%, 0.35%, 0.37%, 0.39%, 0.41%, 0.43%, 0.45%, 0.47%, 0.49%, 0.51%, 0.53%, 0.55%, 0.57%, 0.59%, 0.61%, 0.63%, 0.65%, 0.67%, 0.69%, 0.71%, 0.73%, 0.75%, 0.77%, 0.79%, 0.81%, 0.83%, 0.85%, 0.87%, 0.89%, 0.91%, 0.93%, 0.95%, 0.97%, 0.99%, 1.01%, 1.03%, 1.05%, 1.07%, 1.09%, 1.11%, 1.13%, 1.15%, 1.17%, 1.19%, 1.21%, 1.23%, 1.25%, 1.27%, 1.29%, 1.31%, 1.33%, 1.35%, 1.37%, 1.39%, 1.41%, 1.43%, 1.45%, 1.47%, 1.49%, 1.51%, 1.53%, 1.55%, 1.57%, 1.59%, 1.61%, 1.63%, 1.65%, 1.67%, 1.69%, 1.71%, 1.73%, 1.75%, 1.77%, 1.79%, 1.81%, 1.83%, 1.85%, 1.87%, 1.89%, 1.91%, 1.93%, 1.95%, 1.97%, 1.99%, 2.01%, 2.03%, 2.05%, 2.07%, 2.09%, 2.11%, 2.13%, 2.15%, 2.17%, 2.19%, 2.21%, 2.23%, 2.25%, 2.27%, 2.29%, 2.31%, 2.33%, 2.35%, 2.37%, 2.39%, 2.41%, 2.43%, 2.45%, 2.47%, or 2.49% by weight of the composition. In some embodiments, the at least one active agent is present in an amount of 0.1%, 0.2%, 0.3%, 0.4%, 0.5%, 0.6%, 0.7%, 0.8%, 0.9%, 1%, 1.1%, or 1.2% by weight of the composition.

In some embodiments, the at least one active agent is present in an amount ranging from 0.01% to 1% by weight of the composition. In some embodiments, the at least one active agent is present in an amount ranging from 0.3% to 0.6% by weight of the composition. In some embodiments, the at least one active agent is present in an amount ranging from 0.4% to 0.5% by weight of the composition. In some embodiments, the at least one active agent is present in an amount ranging from 0.1% to 0.5% by weight of the composition. In some embodiments, the at least one active agent is present in an amount of 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, or 0.5% by weight of the composition. In some embodiments, the at least one active agent is present in an amount ranging from 0.1% to 0.3% by weight of the composition.

In some embodiments, the at least one active agent is present in an amount of 0.01%, 0.02%, 0.03%, 0.04%, 0.05%, 0.06%, 0.07%, 0.08%, 0.09%, 0.1%, 0.12%, 0.14%, 0.16%, 0.18%, 0.2%, 0.22%, 0.24%, 0.26%, 0.28%, 0.3%, 0.32%, 0.34%, 0.36%, 0.38%, 0.4%, 0.42%, 0.44%, 0.48%, 0.5%, 0.52%, 0.54%, 0.56%, 0.58%, 0.6%, 0.62%, 0.64%, 0.66%, 0.68%, 0.7%, 0.72%, 0.74%, 0.76%, 0.78%, 0.8%, 0.82%, 0.84%, 0.86%, 0.88%, 0.9%, 0.92%, 0.94%, 0.96%, 0.98%, or 1% by weight of the composition.

In some embodiments, the at least one active agent is present in an amount of 0.08% by weight of the composition. In some embodiments, the at least one active agent is present in an amount of 0.1% by weight of the composition. In some embodiments, the at least one active agent is present in an amount of 0.12% by weight of the composition. In some embodiments, the at least one active agent is present in an amount of 0.14% by weight of the composition. In some embodiments, the at least one active agent is present in an amount of 0.16% by weight of the composition. In some embodiments, the at least one active agent is present in an amount of 0.18% by weight of the composition. In some embodiments, the at least one active agent is present in an amount of 0.2% by weight of the composition. In some embodiments, the at least one active agent is present in an amount of 0.22% by weight of the composition. In some embodiments, the at least one active agent is present in an amount of 0.24% by weight of the composition. In some embodiments, the at least one active agent is present in an amount of 0.26% by weight of the composition. In some embodiments, the at least one active agent is present in an amount of 0.36% by weight of the composition. In some embodiments, the at least one active agent is present in an amount of 0.38% by weight of the composition. In some embodiments, the at least one active agent is present in an amount of 0.4% by weight of the composition. In some embodiments, the at least one active agent is present in an amount of 0.42% by weight of the composition. In some embodiments, the at least one active agent is present in an amount of 0.44% by weight of the composition. In some embodiments, the at least one active agent is present in an amount of 0.46% by weight of the composition. In some embodiments, the at least one active agent is present in an amount of 0.48% by weight of the composition. In some embodiments, the at least one active agent is present in an amount of 0.5% by weight of the composition.

At Least One Solvent

In some embodiments, the composition further comprises at least one solvent. In some embodiments, the at least one solvent is chosen from water, ethanol, isopropanol, polyethylene glycols, and propylene glycols.

In some embodiments, the at least one solvent is water. In some embodiments, the at least one solvent is ethanol. In some embodiments, the at least one solvent is isopropanol. In some embodiments, the at least one solvent is chosen from polyethylene glycols. In some embodiments, the at least one solvent is chosen from propylene glycols. In some embodiments, the polyethylene glycols are chosen from polyethylene glycol 300, polyethylene glycol 400, polyethylene glycol 500, and polyethylene glycol 600.

In some embodiments, the at least one solvent is present in an amount of qs 100% by weight of the composition.

At Least One Tonicity Agent

In some embodiments, the composition further comprises at least one tonicity agent. Without being bound by any theory, in some embodiments, at least one tonicity agent may be added to modulate the solute concentration of a composition.

In some embodiments, the at least one tonicity agent is chosen from dextrose, sorbitol, lactose, mannitol, sodium chloride, potassium chloride, and glycerol. In some embodiments, the at least one tonicity agent is chosen from sodium chloride and potassium chloride. In some embodiments, the at least one tonicity agent is sodium chloride and potassium chloride.

In some embodiments, the at least one tonicity agent is dextrose. In some embodiments, the at least one tonicity agent is sorbitol. In some embodiments, the at least one tonicity agent is lactose. In some embodiments, the at least one tonicity agent is mannitol. In some embodiments, the at least one tonicity agent is sodium chloride. In some embodiments, the at least one tonicity agent is potassium chloride. In some embodiments, the at least one tonicity agent is glycerol.

In some embodiments, the at least one tonicity agent is present in an amount up to 5% by weight of the composition. In some embodiments, the at least one tonicity agent is present in an amount up to 4% by weight of the composition. In some embodiments, the at least one tonicity agent is present in an amount up to 3% by weight of the composition. In some embodiments, the at least one tonicity agent is present in an amount of up to 2.5% by weight of the composition. In some embodiments, the at least one tonicity agent is present in an amount of up to 2% by weight of the composition. In some embodiments, the at least one tonicity agent is present in an amount of up to 1.5% by weight of the composition. In some embodiments, the at least one tonicity agent is present in an amount of up to 1% by weight of the composition. In some embodiments, the at least one tonicity agent is present in an amount of up to 0.5% by weight of the composition.

In some embodiments, the at least one tonicity agent is present in an amount ranging from 0.25% to 3% by weight of the composition. In some embodiments, the at least one tonicity agent is present in an amount ranging from 0.25% to 2.5% by weight of the composition. In some embodiments, the at least one tonicity agent is present in an amount ranging from 0.5% to 2% by weight of the composition. In some embodiments, the at least one tonicity agent is present in an amount ranging from 0.5% to 1.5% by weight of the composition.

In some embodiments, the at least one tonicity agent is present in an amount of 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, or 0.9% by weight of the composition. In some embodiments, the at least one tonicity agent is present in an amount of 0.72%, 0.74%, 0.76%, 0.78%, 0.8%, 0.82%, or 0.84% by weight of the composition. In some embodiments, the at least one tonicity agent is present in an amount of 0.72% by weight of the composition. In some embodiments, the at least one tonicity agent is present in an amount of 0.74% by weight of the composition. In some embodiments, the at least one tonicity agent is present in an amount of 0.76% by weight of the composition. In some embodiments, the at least one tonicity agent is present in an amount of 0.78% by weight of the composition. In some embodiments, the at least one tonicity agent is present in an amount of 0.8% by weight of the composition. In some embodiments, the at least one tonicity agent is present in an amount of 0.82% by weight of the composition. In some embodiments, the at least one tonicity agent is present in an amount of 0.84% by weight of the composition.

At Least One Buffer

In some embodiments, the composition further comprises at least one buffer. Without being bound by any theory, in some embodiments, at least one buffer may be added to maintain a desired pH or pH range.

In some embodiments, the at least one buffer is chosen from a potassium salt, a sodium salt, and maleic acid. In some embodiments, the at least one buffer is a sodium salt. In some embodiments, the at least one buffer is a potassium salt. In some embodiments, the at least one buffer is maleic acid. In some embodiments, the at least one buffer comprises a potassium salt and a sodium salt.

In some embodiments, the potassium salt is chosen from potassium phosphate, potassium citrate, potassium acetate, potassium lactate, and potassium tartrate. In some embodiments, the sodium salt is chosen from sodium phosphate, sodium citrate, sodium acetate, sodium lactate, and sodium tartrate. In some embodiments, the at least one buffer is chosen from potassium phosphate and sodium phosphate. In some embodiments, the at least one buffer comprises potassium phosphate and sodium phosphate.

In some embodiments, the potassium phosphate is chosen from potassium phosphate monobasic, potassium phosphate dibasic, and potassium phosphate tribasic. In some embodiments, the potassium phosphate is chosen from potassium phosphate monobasic and potassium phosphate dibasic. In some embodiments, the potassium phosphate is potassium phosphate monobasic. In some embodiments, the potassium phosphate is potassium phosphate dibasic. In some embodiments, the potassium phosphate is potassium phosphate tribasic.

In some embodiments, the sodium phosphate is chosen from sodium phosphate monobasic, sodium phosphate dibasic, and sodium phosphate tribasic. In some embodiments, the sodium phosphate is chosen from sodium phosphate monobasic and sodium phosphate dibasic. In some embodiments, the sodium phosphate is sodium phosphate monobasic. In some embodiments, the sodium phosphate is sodium phosphate dibasic. In some embodiments, the sodium phosphate is sodium phosphate tribasic.

In some embodiments, the at least one buffer is chosen from sodium phosphate dibasic and potassium phosphate monobasic. In some embodiments, the at least one buffer is sodium phosphate dibasic and potassium phosphate monobasic.

In some embodiments, the at least one buffer is anhydrous.

In some embodiments, the at least one buffer is present in an amount of up to 2.5% by weight of the composition. In some embodiments, the at least one buffer is present in an amount of up to 2% by weight of the composition. In some embodiments, the at least one buffer is present in an amount of up to 1.5% by weight of the composition. In some embodiments, the at least one buffer is present in an amount of up to 1% by weight of the composition. In some embodiments, the at least one buffer is present in an amount of up to 0.5% by weight of the composition.

In some embodiments, the at least one buffer is present in an amount ranging from 0.05% to 0.4% by weight of the composition. In some embodiments, the at least one buffer is present in an amount of 0.06%, 0.08%, 0.1%, 0.12%, 0.14%, 0.16%, 0.18% 0.2%, 0.22%, 0.24%, 0.26%, 0.28%, or 0.3% by weight of the composition. In some embodiments, the at least one buffer is present in an amount ranging from 0.1% to 0.2% by weight of the composition. In some embodiments, the at least one buffer is present in an amount ranging from 0.08% to 0.16% by weight of the composition. In some embodiments, the at least one buffer is present in an amount of 0.1% by weight of the composition. In some embodiments, the at least one buffer is present in an amount of 0.11% by weight of the composition. In some embodiments, the at least one buffer is present in an amount of 0.12% by weight of the composition. In some embodiments, the at least one buffer is present in an amount of 0.13% by weight of the composition. In some embodiments, the at least one buffer is present in an amount of 0.14% by weight of the composition. In some embodiments, the at least one buffer is present in an amount of 0.15% by weight of the composition. In some embodiments, the at least one buffer is present in an amount of 0.16% by weight of the composition. In some embodiments, the at least one buffer is present in an amount of 0.17% by weight of the composition. In some embodiments, the at least one buffer is present in an amount of 0.18% by weight of the composition. In some embodiments, the at least one buffer is present in an amount of 0.19% by weight of the composition. In some embodiments, the at least one buffer is present in an amount of 0.2% by weight of the composition.

In some embodiments, the at least one buffer comprises sodium phosphate in an amount ranging from 0.01% to 0.03% by weight of the composition and potassium phosphate in an amount ranging from 0.1% to 0.14% by weight of the composition.

At Least One pH Modulation Agent

In some embodiments, the composition further comprises at least one pH modulation agent. Without being bound by any theory, in some embodiments, at least one pH modulation agent may be added to achieve a desired pH of the composition.

In some embodiments, the at least one pH modulation agent is chosen from acetic acid, carbonic acid, citric acid, sodium bicarbonate, and sodium hydroxide. In some embodiments, the at least one pH modulation agent is acetic acid. In some embodiments, the at least one pH modulation agent is carbonic acid. In some embodiments, the at least one pH modulation agent is citric acid. In some embodiments, the at least one pH modulation agent is sodium bicarbonate. In some embodiments, the at least one pH modulation agent is sodium hydroxide.

In some embodiments, the at least one pH modulation agent is chosen from acetic acid and sodium hydroxide. In some embodiments, the at least one pH modulation agent is acetic acid and sodium hydroxide.

In some embodiments, the at least one pH modulation agent is present in an amount of qs to desired pH.

At Least One Viscosity Agent

In some embodiments, the composition further comprises at least one viscosity agent. Without being bound by any theory, in some embodiments, at least one viscosity agent may be added to achieve a desired viscosity or thickness of the composition.

In some embodiments, the at least one viscosity agent is chosen from gelatin, methylcellulose, hydroxypropyl cellulose, hydroxypropyl methylcellulose, hydroxyethyl cellulose, hydroxypropylmethyl cellulose, methylhydroxyethyl cellulose, methylhydroxypropyl cellulose, hydroxyethylcarboxymethyl cellulose, carboxymethyl cellulose, carboxymethylhydroxyethyl cellulose, and sodium carboxymethylcellulose. In some embodiments, the at least one viscosity agent is chosen from methylcellulose, sodium carboxymethylcellulose, and hydroxypropyl cellulose.

In some embodiments, the at least one viscosity agent is gelatin. In some embodiments, the at least one viscosity agent is methylcellulose. In some embodiments, the at least one viscosity agent is hydroxypropyl cellulose. In some embodiments, the at least one viscosity agent is hydroxypropyl methylcellulose. In some embodiments, the at least one viscosity agent is hydroxyethyl cellulose. In some embodiments, the at least one viscosity agent is hydroxypropylmethyl cellulose. In some embodiments, the at least one viscosity agent is methylhydroxyethyl cellulose. In some embodiments, the at least one viscosity agent is methylhydroxypropyl cellulose. In some embodiments, the at least one viscosity agent is hydroxyethylcarboxymethyl cellulose. In some embodiments, the at least one viscosity agent is carboxymethyl cellulose. In some embodiments, the at least one viscosity agent is carboxymethylhydroxyethyl cellulose. In some embodiments, the at least one viscosity agent is sodium carboxymethylcellulose.

In some embodiments, the at least one viscosity agent is present in an amount of 2.5% or less by weight of the composition. In some embodiments, the at least one viscosity agent is present in an amount of 2% or less by weight of the composition. In some embodiments, the at least one viscosity agent is present in an amount of 1.5% or less by weight of the composition. In some embodiments, the at least one viscosity agent is present in an amount of 1% or less by weight of the composition. In some embodiments, the at least one viscosity agent is present in an amount of 0.5% or less by weight of the composition.

In some embodiments, the at least one viscosity agent is present in an amount ranging from 0.05% to 1% by weight of the composition. In some embodiments, the at least one viscosity agent is present in an amount ranging from 0.1% to 0.5% by weight of the composition. In some embodiments, the at least one viscosity agent is present in an amount ranging from 0.1% to 0.3% by weight of the composition. In some embodiments, the at least one viscosity agent is present in an amount of 0.15%, 0.16%, 0.17%, 0.18%, 0.19%, 0.2%, 0.21%, 0.22%, 0.23%, 0.24%, or 0.25% by weight of the composition.

In some embodiments, the at least one viscosity agent is present in an amount of 0.15% by weight of the composition. In some embodiments, the at least one viscosity agent is present in an amount of 0.16% by weight of the composition. In some embodiments, the at least one viscosity agent is present in an amount of 0.17% by weight of the composition. In some embodiments, the at least one viscosity agent is present in an amount of 0.18% by weight of the composition. In some embodiments, the at least one viscosity agent is present in an amount of 0.19% by weight of the composition. In some embodiments, the at least one viscosity agent is present in an amount of 0.2% by weight of the composition. In some embodiments, the at least one viscosity agent is present in an amount of 0.21% by weight of the composition. In some embodiments, the at least one viscosity agent is present in an amount of 0.22% by weight of the composition. In some embodiments, the at least one viscosity agent is present in an amount of 0.23% by weight of the composition. In some embodiments, the at least one viscosity agent is present in an amount of 0.24% by weight of the composition. In some embodiments, the at least one viscosity agent is present in an amount of 0.25% by weight of the composition.

At Least One Penetration Enhancer

In some embodiments, the composition further comprises at least one penetration enhancer. Without being bound by any theory, in some embodiments, at least one penetration enhancer may be added to increase the amount of the at least one active agent delivered to the desired location.

In some embodiments, the at least one penetration enhancer is chosen from benzyl alcohol, diethylene glycol monoethyl ether, caprylic acid, and sodium oleate. In some embodiments, the at least one penetration enhancer is benzyl alcohol. In some embodiments, the at least one penetration enhancer is diethylene glycol monoethyl ether. In some embodiments, the at least one penetration enhancer is caprylic acid. In some embodiments, the at least one penetration enhancer is sodium oleate.

In some embodiments, the at least one penetration enhancer is present in an amount ranging from 0.01% to 1% by weight of the composition. In some embodiments, the at least one penetration enhancer is present in an amount ranging from 0.01% to 0.5% by weight of the composition. In some embodiments, the at least one penetration enhancer is present in an amount ranging from 0.05% to 0.25% by weight of the composition. In some embodiments, the at least one penetration enhancer is present in an amount of 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, or 1% by weight of the composition.

At Least One Stabilizing Agent

In some embodiments, the composition further comprises at least one stabilizing agent. Without being bound by any theory, in some embodiments, at least one stabilizing agent may be added to retard or completely prevent degradation of the at least one active agent and/or the stabilizing agent may retard or completely prevent the appearance of impurities in the composition.

In some embodiments, the at least one stabilizing agent is chosen from ascorbic acid, butylated hydroxytoluene, citric acid, benzoic acid, and sodium metabisulfite. In some embodiments, the at least one stabilizing agent is ascorbic acid. In some embodiments, the at least one stabilizing agent is butylated hydroxytoluene. In some embodiments, the at least one stabilizing agent is citric acid. In some embodiments, the at least one stabilizing agent is benzoic acid. In some embodiments, the at least one stabilizing agent is sodium metabisulfite.

In some embodiments, the at least one stabilizing agent is present in an amount ranging from 0.005% to 1% by weight of the composition. In some embodiments, the at least one stabilizing agent is present in an amount ranging from 0.005% to 0.25%, 0.5%, 0.75%, or 1% by weight of the composition.

In some embodiments, the at least one stabilizing agent is present in an amount ranging from 0.01% to 1% by weight of the composition. In some embodiments, the at least one stabilizing agent is present in an amount ranging from 0.01% to 0.25%, 0.5%, 0.75%, or 1% by weight of the composition

In some embodiments, the at least one stabilizing agent is present in an amount ranging from 0.1% to 1% by weight of the composition. In some embodiments, the at least one stabilizing agent is present in an amount ranging from 0.1% to 0.25%, 0.5%, 0.75%, or 1% by weight of the composition.

In some embodiments, the at least one stabilizing agent is present in an amount of 0.005%, 0.01%, 0.025%, 0.05%, 0.1%, 0.15%, 0.2%, 0.25%, 0.3%, 0.4%, 0.5%, 0.75%, or 1% by weight of the composition. In some embodiments, the at least one stabilizing agent is present in an amount of 0.005% by weight of the composition. In some embodiments, the at least one stabilizing agent is present in an amount of 0.01% by weight of the composition. In some embodiments, the at least one stabilizing agent is present in an amount of 0.025% by weight of the composition. In some embodiments, the at least one stabilizing agent is present in an amount of 0.05% by weight of the composition. In some embodiments, the at least one stabilizing agent is present in an amount of 0.1% by weight of the composition. In some embodiments, the at least one stabilizing agent is present in an amount of 0.15% by weight of the composition. In some embodiments, the at least one stabilizing agent is present in an amount of 0.2% by weight of the composition. In some embodiments, the at least one stabilizing agent is present in an amount of 0.25% by weight of the composition. In some embodiments, the at least one stabilizing agent is present in an amount of 0.3% by weight of the composition. In some embodiments, the at least one stabilizing agent is present in an amount of 0.4% by weight of the composition. In some embodiments, the at least one stabilizing agent is present in an amount of 0.5% by weight of the composition. In some embodiments, the at least one stabilizing agent is present in an amount of 0.75% by weight of the composition. In some embodiments, the at least one stabilizing agent is present in an amount of 1% by weight of the composition.

At Least One Solubilizing Agent

In some embodiments, the composition further comprises at least one solubilizing agent. Without being bound by any theory, in some embodiments, at least one solubilizing agent may be added to increase the solubility of the at least one active agent in a vehicle, e.g., water, by forming, e.g., an emulsion.

In some embodiments, the at least one solubilizing agent is chosen from tocopherols, fixed oils, soybean oil, PEG-15 hydroxystearates, polysorbate 20, polysorbate 80, 2-hydroxypropyl-β-cyclodextrin, and γ-cyclodextrin. In some embodiments, the solubilizing agent is chosen from tocopherols. In some embodiments, the solubilizing agent is chosen from fixed oils. In some embodiments, the solubilizing agent is chosen from PEG-15 hydroxystearates. In some embodiments, the solubilizing agent is polysorbate 20. In some embodiments, the solubilizing agent is polysorbate 80. In some embodiments, the solubilizing agent is 2-hydroxypropyl-β-cyclodextrin. In some embodiments, the solubilizing agent is γ-cyclodextrin.

In some embodiments, fixed oils are chosen from corn oil, cottonseed oil, peanut oil, and sesame oil.

In some embodiments, the at least one solubilizing agent is present in an amount ranging from 0.005% to 10% by weight of the composition. In some embodiments, the at least one solubilizing agent is present in an amount ranging from 0.005% to 0.1%, 0.25%, 0.5%, 1%, 1.5%, 2.5%, 5%, 7.5%, or 10% by weight of the composition. In some embodiments, the at least one solubilizing agent is present in an amount ranging from 0.1% to 10% by weight of the composition. In some embodiments, the at least one solubilizing agent is present in an amount ranging from 0.1% to 0.25%, 0.5%, 1%, 1.5%, 2.5%, 5%, 7.5%, or 10% by weight of the composition.

In some embodiments, the at least one solubilizing agent is present in an amount ranging from 1% to 10% by weight of the composition. In some embodiments, the at least one solubilizing agent is present in an amount ranging from 1% to 1.5%, 2.5%, 5%, 7.5%, or 10% by weight of the composition.

In some embodiments, the at least one solubilizing agent is present in an amount of 0.005%, 0.01%, 0.025%, 0.05%, 0.1%, 0.15%, 0.2%, 0.25%, 0.3%, 0.4%, 0.5%, 0.75%, 1%, 1.5%, 2%, 2.5%, 5%, 7.5%, or 10% by weight of the composition. In some embodiments, the at least one solubilizing agent is present in an amount of 0.005% by weight of the composition. In some embodiments, the at least one solubilizing agent is present in an amount of 0.01% by weight of the composition. In some embodiments, the at least one solubilizing agent is present in an amount of 0.025% by weight of the composition. In some embodiments, the at least one solubilizing agent is present in an amount of 0.05% by weight of the composition. In some embodiments, the at least one solubilizing agent is present in an amount of 0.1% by weight of the composition. In some embodiments, the at least one solubilizing agent is present in an amount of 0.15% by weight of the composition. In some embodiments, the at least one solubilizing agent is present in an amount of 0.2% by weight of the composition. In some embodiments, the at least one solubilizing agent is present in an amount of 0.25% by weight of the composition. In some embodiments, the at least one solubilizing agent is present in an amount of 0.3% by weight of the composition. In some embodiments, the at least one solubilizing agent is present in an amount of 0.4% by weight of the composition. In some embodiments, the at least one solubilizing agent is present in an amount of 0.5% by weight of the composition. In some embodiments, the at least one solubilizing agent is present in an amount of 0.75% by weight of the composition. In some embodiments, the at least one solubilizing agent is present in an amount of 1% by weight of the composition. In some embodiments, the at least one solubilizing agent is present in an amount of 1.5% by weight of the composition. In some embodiments, the at least one solubilizing agent is present in an amount of 2% by weight of the composition. In some embodiments, the at least one solubilizing agent is present in an amount of 2.5% by weight of the composition. In some embodiments, the at least one solubilizing agent is present in an amount of 5% by weight of the composition. In some embodiments, the at least one solubilizing agent is present in an amount of 7.5% by weight of the composition. In some embodiments, the at least one solubilizing agent is present in an amount of 10% by weight of the composition.

At Least One Encapsulation Agent

In some embodiments, the composition further comprises at least one encapsulation agent. Without being bound by any theory, in some embodiments, at least one encapsulation agent may be added to improve delivery, stability, and/or solubility of the at least one active agent by, e.g., encapsulating the at least active agent in a liposome or lipid particle.

In some embodiments, the at least one encapsulation agent is chosen from 1,2-dimyristoyl-sn-glycero-phosphocholine, 1,2-distearoyl-sn-glycero-3-(phosphor-rac-(1-glycerol)), 1,2-distearoyl-sn-glycero-3-phosphocholine, cholesterol, DL-dipalmitoylphosphatidylglycerol, sodium N-(carbonyl-methoxypolyethylene glycol 2000)-1,2-distearoyl-sn-glycero-3-phosphoethanolamine, sodium N-(carbonyl-methoxypolyethylene glycol 2000)-distearoyl-glycerophosphoethanolamine, DL-distearoylphosphatidylcholine, egg phospholipids, and hydrogenated soybean lecithin.

In some embodiments, the at least one encapsulation agent is 1,2-dimyristoyl-sn-glycero-phosphocholine. In some embodiments, the at least one encapsulation agent is 1,2-distearoyl-sn-glycero-3-(phosphor-rac-(1-glycerol)). In some embodiments, the at least one encapsulation agent is 1,2-distearoyl-sn-glycero-3-phosphocholine. In some embodiments, the at least one encapsulation agent is cholesterol. In some embodiments, the at least one encapsulation agent is DL-dipalmitoylphosphatidylglyce-rol. In some embodiments, the at least one encapsulation agent is sodium N-(carbonyl-methoxypolyethylene glycol 2000)-1,2-distearoyl-sn-glycero-3-phosphoethanolamine. In some embodiments, the at least one encapsulation agent is sodium N-(carbonyl-methoxypolyethylene glycol 2000)-distearoyl-glycerophosphoethanolamine. In some embodiments, the at least one encapsulation agent is DL-distearoylphosphatidylcholine. In some embodiments, the at least one encapsulation agent is an egg phospholipid. In some embodiments, the at least one encapsulation agent is hydrogenated soybean lecithin.

In some embodiments, the at least one encapsulation agent is present in an amount ranging from 0.005% to 10% by weight of the composition. In some embodiments, the at least one encapsulation agent is present in an amount ranging from 0.005% to 0.1%, 0.25%, 0.5%, 1%, 1.5%, 2.5%, 5%, 7.5%, or 10% by weight of the composition. In some embodiments, the at least one encapsulation agent is present in an amount ranging from 0.1% to 10% by weight of the composition. In some embodiments, the at least one encapsulation agent is present in an amount ranging from 0.1% to 0.25%, 0.5%, 1%, 1.5%, 2.5%, 5%, 7.5%, or 10% by weight of the composition.

In some embodiments, the at least one encapsulation agent is present in an amount ranging from 1% to 10% by weight of the composition. In some embodiments, the at least one encapsulation agent is present in an amount ranging from 1% to 1.5%, 2.5%, 5%, 7.5%, or 10% by weight of the composition.

In some embodiments, the at least one encapsulation agent is present in an amount of 0.005%, 0.01%, 0.025%, 0.05%, 0.1%, 0.15%, 0.2%, 0.25%, 0.3%, 0.4%, 0.5%, 0.75%, 1%, 1.5%, 2%, 2.5%, 5%, 7.5%, or 10% by weight of the composition. In some embodiments, the at least one encapsulation agent is present in an amount of 0.005% by weight of the composition. In some embodiments, the at least one encapsulation agent is present in an amount of 0.01% by weight of the composition. In some embodiments, the at least one encapsulation agent is present in an amount of 0.025% by weight of the composition. In some embodiments, the at least one encapsulation agent is present in an amount of 0.05% by weight of the composition. In some embodiments, the at least one encapsulation agent is present in an amount of 0.1% by weight of the composition. In some embodiments, the at least one encapsulation agent is present in an amount of 0.15% by weight of the composition. In some embodiments, the at least one encapsulation agent is present in an amount of 0.2% by weight of the composition. In some embodiments, the at least one encapsulation agent is present in an amount of 0.25% by weight of the composition. In some embodiments, the at least one encapsulation agent is present in an amount of 0.3% by weight of the composition. In some embodiments, the at least one encapsulation agent is present in an amount of 0.4% by weight of the composition. In some embodiments, the at least one encapsulation agent is present in an amount of 0.5% by weight of the composition. In some embodiments, the at least one encapsulation agent is present in an amount of 0.75% by weight of the composition. In some embodiments, the at least one encapsulation agent is present in an amount of 1% by weight of the composition. In some embodiments, the at least one encapsulation agent is present in an amount of 1.5% by weight of the composition. In some embodiments, the at least one encapsulation agent is present in an amount of 2% by weight of the composition. In some embodiments, the at least one encapsulation agent is present in an amount of 2.5% by weight of the composition. In some embodiments, the at least one encapsulation agent is present in an amount of 5% by weight of the composition. In some embodiments, the at least one encapsulation agent is present in an amount of 7.5% by weight of the composition. In some embodiments, the at least one encapsulation agent is present in an amount of 10% by weight of the composition.

At Least One Imaging Agent

In some embodiments, the composition further comprises at least one imaging agent. In some embodiments, the at least one imaging agent is a radiographic contrast agent. In some embodiments, when at least one imaging agent is present in the composition, the composition further comprising at least one imaging agent is formed by combining a composition comprising at least one imaging agent and a composition comprising at least one active agent. In some embodiments, the composition comprising at least one active agent is a composition described herein.

In some embodiments, when at least one imaging agent is present in the composition, the composition further comprising at least one imaging agent is formed by combining a composition comprising at least one imaging agent and a composition comprising at least one active agent prior to use of the composition comprising at least one active agent and further comprising at least one imaging agent. In some embodiments, when at least one imaging agent is present in the composition, the composition further comprising at least one imaging agent is formed by combining a composition comprising at least one imaging agent and a composition comprising at least one active agent 15 seconds to 24 hours prior to use of the composition comprising at least one active agent and further comprising at least one imaging agent. In some embodiments, when at least one imaging agent is present in the composition, the composition further comprising at least one imaging agent is formed by combining a composition comprising at least one imaging agent and a composition comprising at least one active agent 15 seconds to 6 hours prior to use of the composition comprising at least one active agent and further comprising at least one active agent. In some embodiments, when at least one imaging agent is present in the composition, the composition further comprising at least one imaging agent is formed by combining a composition comprising at least one imaging agent and a composition comprising at least one active agent 15 seconds to 1 hour prior to use of the composition comprising at least one active agent and further comprising at least one active agent. In some embodiments, when at least one imaging agent is present in the composition, the composition further comprising at least one imaging agent is formed by combining a composition comprising at least one imaging agent and a composition comprising at least one active agent less than 2 hours prior to use of the composition comprising at least one active agent and further comprising at least one imaging agent.

In some embodiments, when at least one imaging agent is present in the composition, the ratio of the volume of a composition comprising at least one imaging agent to the volume of a composition comprising at least one active agent ranges from 0.5:1 to 2.5:1 prior to combination. In some embodiments, when at least one imaging agent is present in the composition, the ratio of the volume of a composition comprising at least one imaging agent to the volume of a composition comprising at least one active agent ranges from 0.5:1 to 1.5:1 prior to combination. In some embodiments, when at least one imaging agent is present in the composition, the ratio of the volume of a composition comprising at least one imaging agent to the volume of a composition comprising at least one active agent ranges from 0.75:1 to 1.25:1 prior to combination.

In some embodiments, when at least one imaging agent is present in the composition, the ratio of the volume of a composition comprising at least one imaging agent to the volume of a composition comprising at least one active agent is 0.5:1 prior to combination. In some embodiments, when at least one imaging agent is present in the composition, the ratio of the volume of a composition comprising at least one imaging agent to the volume of a composition comprising at least one active agent is 0.75:1 prior to combination. In some embodiments, when at least one imaging agent is present in the composition, the ratio of the volume of a composition comprising at least one imaging agent to the volume of a composition comprising at least one active agent is 1:1 prior to combination. In some embodiments, when at least one imaging agent is present in the composition, the ratio of the volume of a composition comprising at least one imaging agent to the volume of a composition comprising at least one active agent is 1.25:1 prior to combination. In some embodiments, when at least one imaging agent is present in the composition, the ratio of the volume of a composition comprising at least one imaging agent to the volume of a composition comprising at least one active agent is 1.5:1 prior to combination. In some embodiments, when at least one imaging agent is present in the composition, the ratio of the volume of a composition comprising at least one imaging agent to the volume of a composition comprising at least one active agent is 1.75:1 prior to combination. In some embodiments, when at least one imaging agent is present in the composition, the ratio of the volume of a composition comprising at least one imaging agent to the volume of a composition comprising at least one active agent is 2:1 prior to combination. In some embodiments, when at least one imaging agent is present in the composition, the ratio of the volume of a composition comprising at least one imaging agent to the volume of a composition comprising at least one active agent is 2.25:1 prior to combination. In some embodiments, when at least one imaging agent is present in the composition, the ratio of the volume of a composition comprising at least one imaging agent to the volume of a composition comprising at least one active agent is 2.5:1 prior to combination.

In some embodiments, when at least one imaging agent is present in the composition, the molar ratio of the at least one imaging agent to the at least one active agent ranges from 300:1 to 50:1. In some embodiments, when at least one imaging agent is present in the composition, the molar ratio of the at least one imaging agent to the at least one active agent ranges from 250:1 to 75:1. In some embodiments, when at least one imaging agent is present in the composition, the molar ratio of the at least one imaging agent to the at least one active agent ranges from 200:1 to 100:1. In some embodiments, when at least one imaging agent is present in the composition, the molar ratio of the at least one imaging agent to the at least one active agent ranges from 175:1 to 125:1. In some embodiments, when at least one imaging agent is present in the composition, the molar ratio of the at least one imaging agent to the at least one active agent is 158:1.

At Least One Anti-Proliferative Agent

In some embodiments, the composition further comprises at least one anti-proliferative agent. In some embodiments, when at least one anti-proliferative agent is present in the composition, the composition further comprising at least one anti-proliferative agent is formed by combining a composition comprising at least one anti-proliferative agent and a composition comprising at least one active agent. In some embodiments, the composition comprising at least one active agent is a composition described herein.

In some embodiments, when at least one anti-proliferative agent is present in the composition, the composition further comprising at least one anti-proliferative agent is formed by combining a composition comprising at least one anti-proliferative agent and a composition comprising at least one active agent prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent 15 seconds to 24 hours prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent 15 seconds to 6 hours prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent 15 seconds to 1 hour prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent less than 2 hours prior to use.

In some embodiments, the at least one anti-proliferative agent is chosen from paclitaxel, paclitaxel derivatives, rapamycin, rapamycin derivatives, and pharmaceutically acceptable salts thereof. In some embodiments, the at least one anti-proliferative agent is paclitaxel. In some embodiments, the paclitaxel derivatives are chosen from docetaxel, and cabazitaxel. In some embodiments, the at least one anti-proliferative agent is rapamycin. In some embodiments, the rapamycin derivatives are chosen from everolimus, ridaforolimus, tacrolimus, umirolimus, and zotarolimus.

In some embodiments, when at least one anti-proliferative agent is present in the composition, the ratio of the volume of a composition comprising at least one anti-proliferative agent to the volume of a composition comprising at least one active agent ranges from 0.5:1 to 2.5:1 prior to combination. In some embodiments, when at least one anti-proliferative agent is present in the composition, the ratio of the volume of a composition comprising at least one anti-proliferative agent to the volume of a composition comprising at least one active agent ranges from 0.5:1 to 1.5:1 prior to combination. In some embodiments, when at least one anti-proliferative agent is present in the composition, the ratio of the volume of a composition comprising at least one anti-proliferative agent to the volume of a composition comprising at least one active agent ranges from 0.75:1 to 1.25:1 prior to combination.

In some embodiments, when at least one anti-proliferative agent is present in the composition, the ratio of the volume of a composition comprising at least one anti-proliferative agent to the volume of a composition comprising at least one active agent is 0.5:1 prior to combination. In some embodiments, when at least one anti-proliferative agent is present in the composition, the ratio of the volume of a composition comprising at least one anti-proliferative agent to the volume of a composition comprising at least one active agent is 0.75:1 prior to combination. In some embodiments, when at least one anti-proliferative agent is present in the composition, the ratio of the volume of a composition comprising at least one anti-proliferative agent to the volume of a composition comprising at least one active agent is 1:1 prior to combination. In some embodiments, when at least one anti-proliferative agent is present in the composition, the ratio of the volume of a composition comprising at least one anti-proliferative agent to the volume of a composition comprising at least one active agent is 1.25:1 prior to combination. In some embodiments, when at least one anti-proliferative agent is present in the composition, the ratio of the volume of a composition comprising at least one anti-proliferative agent to the volume of a composition comprising at least one active agent is 1.5:1 prior to combination. In some embodiments, when at least one anti-proliferative agent is present in the composition, the ratio of the volume of a composition comprising at least one anti-proliferative agent to the volume of a composition comprising at least one active agent is 1.75:1 prior to combination. In some embodiments, when at least one anti-proliferative agent is present in the composition, the ratio of the volume of a composition comprising at least one anti-proliferative agent to the volume of a composition comprising at least one active agent is 2:1 prior to combination. In some embodiments, when at least one anti-proliferative agent is present in the composition, the ratio of the volume of a composition comprising at least one anti-proliferative agent to the volume of a composition comprising at least one active agent is 2.25:1 prior to combination. In some embodiments, when at least one anti-proliferative agent is present in the composition, the ratio of the volume of a composition comprising at least one anti-proliferative agent to the volume of a composition comprising at least one active agent is 2.5:1 prior to combination.

In some embodiments, when at least one anti-proliferative agent is present in the composition, the molar ratio of the at least one anti-proliferative agent to the at least one active agent ranges from 0.25:4 to 4:0.25. In some embodiments, when at least one anti-proliferative agent is present in the composition, the molar ratio of the at least one anti-proliferative agent to the at least one active agent ranges from 0.75:2 to 2:0.75. In some embodiments, when at least one anti-proliferative agent is present in the composition, the molar ratio of the at least one anti-proliferative agent to the at least one active agent ranges from 0.75:1.5 to 1.25:1.5. In some embodiments, when at least one anti-proliferative agent is present in the composition, the molar ratio of the at least one anti-proliferative agent to the at least one active agent ranges from 0.9:1.3 to 1.1:1.5. In some embodiments, when at least one anti-proliferative agent is present in the composition, the molar ratio of the at least one anti-proliferative agent to the at least one active agent is 1:1.4.

Composition Properties

Composition pH

In some embodiments, the composition has a pH ranging from 4 to 8. In some embodiments, the composition has a pH ranging from 5 to 7. In some embodiments, the composition has a pH ranging from 5.5 to 6.5.

Without being bound by any theory, in some embodiments, a composition having a pH ranging from 5 to 7 may be suitable for intraarterial or intravenous delivery and/or may result in a composition where the at least one active agent does not degrade after a period of time compared to a composition having a pH outside of that range, as described herein below.

In some embodiments, the composition has a pH of 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7. In some embodiments, the composition has a pH of 5. In some embodiments, the composition has a pH of 5.1. In some embodiments, the composition has a pH of 5.2. In some embodiments, the composition has a pH of 5.3. In some embodiments, the composition has a pH of 5.4. In some embodiments, the composition has a pH of 5.5. In some embodiments, the composition has a pH of 5.6. In some embodiments, the composition has a pH of 5.7. In some embodiments, the composition has a pH of 5.8. In some embodiments, the composition has a pH of 5.9. In some embodiments, the composition has a pH of 6. In some embodiments, the composition has a pH of 6.1. In some embodiments, the composition has a pH of 6.2. In some embodiments, the composition has a pH of 6.3. In some embodiments, the composition has a pH of 6.4. In some embodiments, the composition has a pH of 6.5. In some embodiments, the composition has a pH of 6.6. In some embodiments, the composition has a pH of 6.7. In some embodiments, the composition has a pH of 6.8. In some embodiments, the composition has a pH of 6.9. In some embodiments, the composition has a pH of 7.

Non-Limiting Exemplary Composition Embodiments

In some embodiments, provided herein is a composition comprising at least one active agent; at least one tonicity agent; at least one buffer; and at least one vehicle.

In some embodiments, the composition comprises at least one active agent in an amount ranging from 0.01% to 1% by weight of the composition; at least one tonicity agent in an amount ranging from 0.5% to 1.5% by weight of the composition; at least one buffer in an amount ranging from 0.05% to 0.4% by weight of the composition; and at least one vehicle. In some embodiments, the composition comprises at least one active agent in an amount ranging from 0.01% to 1% by weight of the composition; at least one tonicity agent in an amount ranging from 0.5% to 1.5% by weight of the composition; at least one buffer in an amount ranging from 0.05% to 0.4% by weight of the composition; and at least one vehicle, wherein the composition has a pH of 6. In some embodiments, the composition comprises at least one active agent in an amount ranging from 0.01% to 1% by weight of the composition; at least one tonicity agent in an amount ranging from 0.5% to 1.5% by weight of the composition; at least one buffer in an amount ranging from 0.05% to 0.4% by weight of the composition; and at least one vehicle, wherein the composition has a pH of 6, and wherein the at least one active agent does not degrade 156 weeks after preparation of the composition.

In some embodiments, provided herein is a composition comprising at least one active agent chosen from Compound of Formula (I) and pharmaceutically acceptable salts thereof; at least one tonicity agent chosen from potassium chloride and sodium chloride; at least one buffer chosen from potassium phosphate and sodium phosphate; and at least one vehicle comprising water. In some embodiments, the composition has a pH of 6. In some embodiments, provided herein is a composition comprising at least one active agent chosen from Compound of Formula (I) and pharmaceutically acceptable salts thereof, at least one tonicity agent chosen from potassium chloride and sodium chloride; at least one buffer chosen from potassium phosphate and sodium phosphate; and at least one vehicle comprising water, wherein the composition has a pH of 6, and wherein the at least one active agent does not degrade 156 weeks after preparation of the composition.

In some embodiments, provided herein is a composition comprising at least one active agent chosen from diacetate salts of Compound of Formula (I); at least one tonicity agent chosen from potassium chloride and sodium chloride; at least one buffer chosen from potassium phosphate and sodium phosphate; and at least one vehicle comprising water. In some embodiments, the composition further comprises at least one active agent. In some embodiments, provided herein is a composition comprising at least one active agent chosen from diacetate salts of Compound of Formula (I); at least one tonicity agent chosen from potassium chloride and sodium chloride; at least one buffer chosen from potassium phosphate and sodium phosphate; and at least one vehicle comprising water, wherein the composition has a pH of 6. In some embodiments, the composition further comprises at least one active agent. In some embodiments, provided herein is a composition comprising at least one active agent chosen from diacetate salts of Compound of Formula (I); at least one tonicity agent chosen from potassium chloride and sodium chloride; at least one buffer chosen from potassium phosphate and sodium phosphate; and at least one vehicle comprising water, wherein the composition has a pH of 6, and wherein the at least one active agent does not degrade 156 weeks after preparation of the composition. In some embodiments, degradation is determined by LC. In some embodiments, the composition further comprises at least one active agent. In some embodiments, the composition further comprises at least one anti-proliferative agent.

In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, wherein the composition has a pH of 6. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, wherein the composition has a pH of 6, and wherein the at least one active agent does not degrade 156 weeks after preparation of the composition. In some embodiments, degradation is determined by LC. In some embodiments, the composition further comprises at least one active agent. In some embodiments, the composition further comprises at least one anti-proliferative agent.

In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent. In some embodiments, the composition further comprises at least one anti-proliferative agent. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent. In some embodiments, the composition further comprises at least one anti-proliferative agent. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent, wherein the at least one imaging agent comprises a radiographic contrast agent. In some embodiments, the composition further comprises at least one anti-proliferative agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent, wherein the at least one active agent does not degrade 24 hours after preparation of the composition. In some embodiments, degradation is determined by LC. In some embodiments, the composition further comprises at least one active agent. In some embodiments, the composition further comprises at least one anti-proliferative agent.

In some embodiments, the at least one active agent is chosen from a Compound of Formula (I) and pharmaceutically acceptable salts thereof. In some embodiments, the at least one active agent is a diacetate salt of a Compound of Formula (I).

In some embodiments, the at least one active agent is a diacetate salt of a Compound of Formula (I) present in an amount of 0.01% to 1% by weight of the composition. In some embodiments, the at least one active agent is a diacetate salt of a Compound of Formula (I) present in an amount of 0.01% to 1% by weight of the composition and the at least one tonicity agent is chosen from potassium chloride and sodium chloride. In some embodiments, the at least one active agent is a diacetate salt of a Compound of Formula (I) present in an amount of 0.01% to 1% by weight of the composition and the at least one tonicity agent is chosen from potassium chloride and sodium chloride present in an amount ranging from 0.5% to 1.5% by weight of the composition. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; and the at least one buffer is chosen from sodium phosphate dibasic and potassium phosphate monobasic. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; and the at least one buffer is chosen from sodium phosphate dibasic and potassium phosphate monobasic present in an amount of 0.1% to 0.16% by weight of the composition. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, wherein the composition has a pH of 6.

In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, wherein the pH of the composition is 6.

In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent chosen from methylcellulose, sodium carboxymethylcellulose, and hydroxypropyl cellulose. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent chosen from methylcellulose, sodium carboxymethylcellulose, and hydroxypropyl cellulose in an amount ranging from 0.1% to 0.3% by weight of the composition. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent chosen from methylcellulose, sodium carboxymethylcellulose, and hydroxypropyl cellulose in an amount ranging from 0.1% to 0.3% by weight of the composition, wherein the composition has a pH of 6.

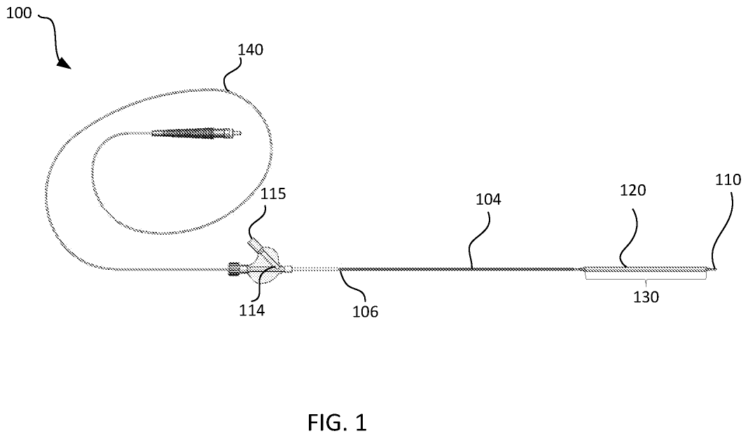

FIG. 1 illustrates an apparatus 100 in accordance with an embodiment of this disclosure. The apparatus 100 having a light fiber 140 incorporated into or used as a catheter shaft 104 that extends from a proximal end 106 to a distal tip 110 of the apparatus 100. The apparatus 100 may be configured for longitudinal movement and positioning within a vessel (e.g. blood vessel) of a subject. In some embodiments, the apparatus 100 may be configured for occlusion of the vessel and treatment of an area of the vessel. For example, the apparatus 100 may be configured for occlusion of a blood vessel and delivery of a drug to the occluded area of the vessel and forming and casting a shape in the vessel, as will be described in more detail below.

The apparatus 100 may include a proximal end connector 114 positioned at the proximal end of the apparatus 100, and the catheter shaft 104 may extend in a distal direction therefrom. The catheter shaft 104 incorporation with the light fiber 140 may define a plurality of lumens that are accessible via a plurality of ports the proximal end connector 114. The plurality of ports 115 may be configured to engage with external sources desirable to communicate with the plurality of lumens. The ports may engage with external sources via a variety of connection mechanisms, including, but not limited to, syringes, over-molding, quick disconnect connectors, latched connections, barbed connections, keyed connections, threaded connections, or any other suitable mechanism for connecting one of the plurality of ports to an external source. Non-limiting examples of external sources may include inflation sources (e.g. saline solutions), gaseous sources, treatment sources (e.g. medication, drugs, or any desirable treatment agents discussed further below), light sources, among others. In some embodiments, apparatus 100 can be used with a guide wire (not shown), via guide wire lumen (not shown), to assist in guiding the catheter shaft 104 to the target area of the vessel.

In some embodiments, a light fiber 140 can be incorporated into catheters used for the apparatus 100. A catheter body may have one or more optically sufficient performing light fibers 140. The light fibers form a continuous light path of the catheter shaft body.

The apparatus 100 may include a distal balloon 120 positioned over a distal segment 130 of the catheter shaft 104 proximal to the distal tip 110. In some embodiments, the distal balloon 120 may be proximally offset from the distal tip 110 a distance between 0 mm and 1 mm, 0 mm and 2 mm, 0 mm and 3 mm, 0 mm and 10 mm, or O and 50 mm. In some embodiments, the distal balloon 120 may inflate to 2 to 10 millimeters (mm) in diameter. In other embodiments, the distal balloon 120 may inflate to 2 to 4 cm in diameter. The distal balloon 120 may have a length of about 0.5 to 1 centimeters (cm), 1 to 2 cm, 1 to 3 cm, or 1 to 5 cm, or 1 to 10 cm, or 1 to 15 cm, or 1 to 20 cm, or 1 to 25 cm, and may take any shape suitable for supporting a wall of a blood vessel of the subject when the non-compliant or semi-compliant balloon is inflated. For example, the distal balloon 120 may expand into a cylindrical shape surrounding the distal segment 130 of the catheter shaft 104. The cylindrical shape may be gradually tapered inward at a proximal end and a distal end of the distal balloon 120, thereby providing a gradually tapered proximal end and distal end of the distal balloon 120 that taper into contact with and become flush with the catheter shaft 104.

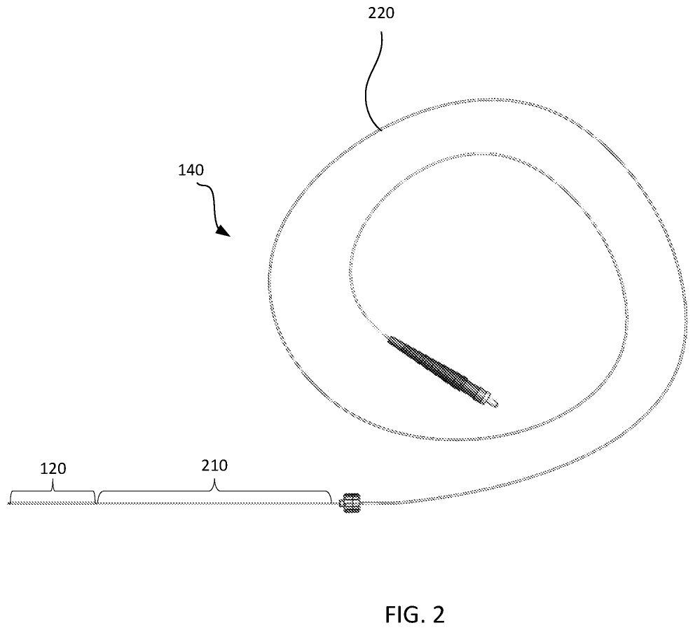

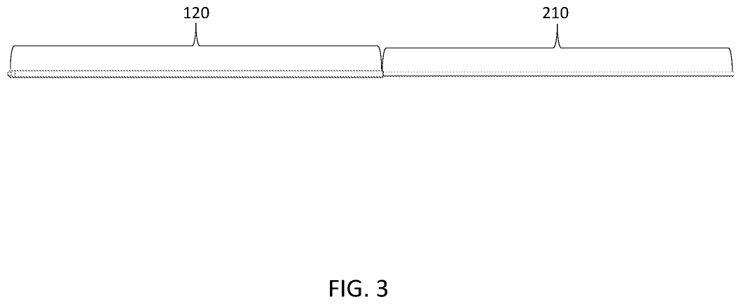

FIGS. 2 and 3 illustrate that the light fiber 140 may include an emission zone, a catheter portion 210, and a protective tubing portion 220. The emission zone can be the distal balloon 120 which includes a light diffuser for activating a NVS agent. The catheter portion 210 is in between the emission zone 120 and the protective tubing portion 220. The catheter portion 210 and the emission zone 120 can be formed by one or more light fibers. The material of the emission zone 120 can be material 1, which may include plastic material such as PMMA. The protective tubing portion 220 and the catheter portion 210 can be made by material 2. In some embodiments, material 1 can be different from material 2. Material 2 can be a type of glass, such as silica; or a flexible plastic, such as nylon, PEBAX, HDPE, LDPE, polyethlyene, polyurethane; or a composite material of plastic with a reinforcement, for example, metal wires, metal coil, plastic fibers. In some embodiments, the distal balloon 120 can be coated with a coating material, for example, NVS, consistent with the disclosed embodiments.

Visible Light

The methods disclosed herein comprise illuminating the at least one active agent with visible light, such as, for example, 450 nm light. The source of visible light may be light supplied to tissue using an optical fiber device. Other wavelengths are possible, including both visible and invisible light, according to the NVS agent formulation.

Accordingly, the apparatus and methods described herein provide the delivery of NVS to a treatment area (e.g. a vessel) and provide restoration to that treatment area using the apparatus or according to the methods described above. The apparatus and method described above provide concurrently scoring the vessel, treating the vessel with one or more drugs (e.g. with Paclitaxel and NVS) with minimal loss to other vessels, scaffolding and casting the vessel, and light activation of the one or more drugs delivered to the treatment area. These advantages can be accomplished utilizing the apparatus and methods described herein.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware and software, but systems and methods consistent with the present disclosure can be implemented as hardware alone. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps and/or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. An apparatus comprising:
a catheter device extending from a proximal section to a distal section,
the distal section having a light diffuser for activating a Natural Vascular Scaffolding (NVS) agent;
a catheter body having one or more optically sufficient performing light fibers,
wherein the one or more light fibers form a continuous light path of the catheter body.

2. The catheter device of claim 1, wherein one or more light fibers include an emission zone, a catheter portion, and a protective tubing portion.

3. The catheter device of claim 2, wherein the emission zone located at the distal section.

4. The catheter device of claim 2, wherein the emission zone can be configured to coat with the NVS agent.

5. The catheter device of claim 2, wherein the emission zone is made of material 1, which can be a type of plastic material, such as PMMA.

6. The catheter device of claim 1, wherein the catheter portion and the protective tubing portion are made of a material 2.

7. The catheter device of claim 6, wherein material 2 is a different material than the material 1.

8. The catheter device of claim 6, wherein material 2 can be a type of glass, a flexible plastic, or a composite material of plastic with a reinforcement, such as, silica, nylon, PEBAX, HDPE, LDPE, polyethylene, polyurethane, metal wires, metal coil, plastic fibers.

9. The catheter device of claim 1, wherein the light fiber in the proximal section is made of a core diameter of 0.25 mm.

10. The catheter device of claim 1, wherein the entire assembly of the catheter formed with light fibers can be electron beam sterilized.

11. A method for sterilizing the catheter device comprising:
providing a catheter, the catheter comprising:
a proximal section to a distal section,
the distal section having a light diffuser for activating a Natural Vascular Scaffolding (NVS) agent; and
a catheter body having one or more optically sufficient performing light fibers,
wherein the one or more light fibers form a continuous light path of the catheter body;
sterilizing the entire assembly of the catheter formed with light fibers with electron beam sterilization.

12. The method for sterilizing the catheter device of claim 11, wherein one or more light fibers include an emission zone, a catheter portion, and a protective tubing portion.

13. The method for sterilizing the catheter device of claim 12, wherein the emission zone located at the distal section.

14. The method for sterilizing the catheter device of claim 12, wherein the emission zone can be configured to coat with the NVS agent.

15. The method for sterilizing the catheter device of claim 12, wherein the emission zone is made of material 1, which can be a type of plastic material, such as PMMA.

16. The method for sterilizing the catheter device of claim 11, wherein the catheter portion and the protective tubing portion are made of a material 2.

17. The method for sterilizing the catheter device of claim 16, wherein material 2 is a different material than the material 1.

18. The method for sterilizing the catheter device of claim 16, wherein material 2 can be a type of glass, a flexible plastic, or a composite material of plastic with a reinforcement, such as, silica, nylon, PEBAX, HDPE, LDPE, polyethylene, polyurethane, metal wires, metal coil, plastic fibers.

19. The method for sterilizing the catheter device of claim 11, wherein the light fiber in the proximal section is preferred to have a smaller core diameter.

20. The method for sterilizing the catheter device of claim 19, wherein the light fiber in the proximal section is preferred to have a core diameter of 0.25 mm.
